(19) United States
(12) Patent Application Publication
(10) Pub. No.: US 2010/0284749 A1
(43) Pub. Date: Nov. 11, 2010

(54) Systems and methods for off-shore energy production and carbon dioxide sequestration

(76) Inventor: Mark E. Capron, Oxnard, CA (US)

Correspondence Address: KOPPEL, PATRICK, HEYBL & DAWSON, 2815 Townsgate Road, SUITE 215, Westlake Village, CA, 91361-5827, US

(21) Appl. No.: 11/985,196

(22) Filed: Nov. 13, 2007

(51) Int. Cl. E02D 29/00; B01D 37/00; C12M 1/00

(52) U.S. Cl. 405/210; 210/767; 435/289.1; 435/41

(57) ABSTRACT

The present invention is directed to aquatic systems and methods for off-shore energy production, particularly to systems and methods for generating large amounts of methane via anaerobic digestion, purifying the methane produced, and sequestering environmentally deleterious by-products such as carbon dioxide. The energy production systems contain one or more flexible, inflatable containers supported by water, at least one of which is an anaerobic digester containing bacteria which can produce energy sources such as methane or hydrogen from aquatic plants or animals. Off-shore energy production facilities supported by water bodies offer many advantages over land-based digesters, including the use of large, available open water bodies as an alternative means of support and the potential for locating the facilities at sites that already contain, or can be easily modified to generate, sufficient amounts of feedstock onsite. In addition, the containers of the present invention can be large enough to provide adequate amounts of energy to support off-shore activities and relatively easy to manufacture and ship to remote production sites. The systems can also be readily adapted to sequester carbon dioxide or replenish feedstocks growing nutrients on site.

### BACKGROUND OF THE INVENTION

1. Field of the Invention

The present invention relates generally to the generation of renewable energy sources and, more particularly, to off-shore systems and methods for the production and purification of energy sources and sequestration of by-products produced thereby.

2. Description of the Related Art

Microorganisms can play a huge role in creating different types of energy by helping to produce usable sources of energy, including hydrogen (H₂) and methane (CH₄). Anaerobic digestion is a biological, bacteria-mediated process that occurs when organic matter is deprived of oxygen. The process exists naturally in water bodies at every depth, in landfills, swamps, aboveground tanks, belowground tanks or ponds, anywhere from a ground surface to miles below the ground surface, in the sediments below water bodies, and in collapsible aboveground containers. It is a very robust process occurring over a wide temperature range, including the full range of ocean temperatures.

Anaerobic digesters can operate with temperatures and pH controlled within a few degrees and a few pH units, or they can operate with widely varying temperature and pH. Some digesters are mixed, some are unmixed, some are plug flow, and some employ multiple chambers. The refinements developed were historically oriented toward the more rapid reduction of waste materials into less harmful constituents of water and carbon dioxide (CO₂). Methane, hydrogen sulfide, and other gases were considered by-products. More rapid reduction allows a smaller container to accomplish the same waste reduction. If the methane by-product was not released to atmosphere, it was typically converted to water and carbon dioxide in a flare. More recently, with higher fossil fuel costs and concern for greenhouse gases, the methane is often burned to produce supplemental electricity.

Both natural and human operated anaerobic digestion processes typically release by-products such as carbon dioxide and other gases into the atmosphere, including methane which is about 20 times more potent than carbon dioxide when released to atmosphere as a greenhouse gas. The greenhouse effect results primarily from human activities such as the burning of fossil fuels, deforestation, agricultural activities, and the use of chemicals such as chlorofluorocarbons and halons and, relatively speaking, non-fossil carbon dioxide released by anaerobic digestion does not contribute significantly to the global warming. The cumulative effect of such emissions over time, however, could be substantial for large, multiple digestion systems, such that not sequestering carbon dioxide or producing energy from the methane released by anaerobic digestion would be a missed opportunity.

Current technologies for reducing greenhouse gases either require energy to capture and sequester carbon dioxide, or produce energy without sequestering carbon dioxide, and it is difficult to reduce the rate of increasing atmospheric carbon dioxide concentrations with either approach alone. For example, about 6% of world energy demand was met with renewable energy in 2000. If renewable energy increased to 60%, one would still need to sequester the carbon dioxide as a by-product of the 40% remaining energy provided by fossil fuels, and sequestering that amount of fossil carbon dioxide using existing resources would probably require new forests covering virtually all of North and South America, as well as fresh water to sustain them. Furthermore, even if those resources were available, forests do not sequester carbon for more than a few centuries and forest sequestering can be undone by fires or the lack of sufficient storage (to prevent release of carbon dioxide due to wood decay).

In light of the environmental damage caused by burning fossil fuels (e.g. the greenhouse effect) and the depletion of easily obtained fossil petroleum, substantial alternative energy sources should be quickly identified and developed. Furthermore, remote facilities without ready access to fossil oil and gas, for example, such as those on the Outer Continental Shelf (OCS), need to produce or support production, transportation, or transmission of energy from alternative energy sources in order to support the activities as such sites. Alternative energy technologies and areas about which industry has expressed a potential interest, and the ability to develop or evaluate in a reasonable time frame, include offshore wind, wave, and ocean current energy capture technologies. Other energy sources, such as solar power and the production of hydrogen, are not expected to be economically viable for the foreseeable future, particularly with respect to off-shore activities such as OCS research, demonstration, or commercial ventures.

Methane is a cost-effective renewable energy source (fuel), and the production of methane and other serviceable biogases by the anaerobic digestion of various organic wastes, particularly sewage sludge organic waste, is well known. Furthermore, the organic feed mixture which provides the substrate for anaerobic biodegradation may actually comprise a wide variety of organic carbon sources ranging from raw sewage sludge to municipal refuse, or biomass material such as plants and crop wastes. Many types of digester designs, feed stocks, mixtures and additives have been proposed to increase the methane yield and to provide greater conversion efficiency of organic materials to useful products, and numerous processes which optimize methane production (both rate of gas production and purity of methane, have been developed over the past millennium.

Although the methane produced as a by-product of terrestrial waste treatment facilities having anaerobic digesters can be used as a supplemental power source for such facilities, large scale production of methane as a commercially viable, alternative energy source requires a system dedicated to that purpose. However, such facilities are typically not cost-effective in that they require significant amounts of land and resources, including enclosures, support structures and a ready supply of the feedstock used to fuel digestion. The scale of energy generation required for cost-effective production, for example, is similar to that needed for generating ethanol, wind energy, solar photovoltaic energy, wave energy, and the like. Significant energy production using typical existing waste treatment technologies can, in fact, be approximately two or more orders of magnitude more than that which could be produced from a global supply of human waste.

Floating waste treatment technologies located on a naturally occurring body of water have therefore been proposed as a more cost-effective alternative for large-scale methane production. The surface of the ocean, for example, can be an essentially free region in which the cost of constructing such a facility might be substantially reduced compared with building a similar facility on land. In addition, the principles of fluid equilibrium can be utilized to reduce the stress upon the containment walls in such a facility, to a level small enough to allow relatively inexpensive and substantially flexible membranes to be used as the barrier separating the waste fluid being processed from the body of water in which a production facility is located. However, the water in the anaerobic digester also accumulates a variety of by-products from the organic and inorganic materials which bacteria don't digest (or digest much more slowly). The predominant by-products are plant nutrients or plant nutrient pre-cursors such as ammonia, phosphorus, and iron which can be utilized productively in other activities, such as fertilizing crops. However, an excess of either undigested waste organic matter or such by-product nutrients can pollute a water body by causing eutrophication of ponds and lakes, and “dead zones” in gulfs and seas. The potential for such detrimental environmental impacts is, in fact, one of the main reasons why water supported containers for processing human or animal waste material are not feasible in many situations. An accidental release of unprocessed waste material could also pose a significant threat to health in many situations.

Information relevant to attempts to address these problems can be found in U.S. Pat. Nos. 7,258,790; 7,186,339; 7,179,387; 7,169,296; 7,137,945; 7,128,777; 7,056,062; 6,984,305; 6,569,332; 5,304,356; 4,944,872; 4,424,064; 4,354,936; 3,981,803; 3,981,800 as well as U.S. Patent Application Nos. 2007/0028848 and 2006/0178547. However, each one of these references suffers from one or more of the following disadvantages:

(1) the method does not produce energy or sequester carbon;

(2) the method improves the value or purity of a gaseous energy source, but does not produce the gas itself;

(3) the method of energy production or carbon sequestering disclosed would be impractical for terrestrial use on a large, commercial scale;

(4) the flexible enclosures disclosed require external support;

(5) the digester volumes required for organic waste reduction are small relative to the tremendous volumes needed for renewable energy production and/or associated carbon dioxide sequestering;

(6) the process disclosed for generating a renewable energy source itself requires energy supplied by electrical currents and systems for maintaining such currents, which can significantly increase both construction and maintenance costs;

(7) aquatic feedstock must be transported to a remote terrestrial digester, or non-aquatic feedstock must be transported from a remote terrestrial source to an aquatic digester, either of which could require construction of lengthy or expensive transport means such as pipelines or equiducts;

(8) waste feedstock, including wastewater sludge or biosolids, is not natural to the aquatic environment and an accidental or deliberate release of such feedstock into a water body could pose a serious health or environmental hazard;

(9) the technologies or methods for producing renewable fuel or sequestering carbon dioxide do not replace sufficient feedstock growing nutrients to allow sustained operation of either process;

(10) carbon dioxide or other greenhouse gas by-products are released into the atmosphere;

(11) the technologies or methods for producing renewable fuel do not sequester carbon dioxide as a by-product of fuel production;

(12) the technologies or methods for sequestering carbon dioxide do not produce a renewable fuel in quantities that make the sequestering process cost effective;

(13) once released to atmosphere, the resulting low atmospheric carbon dioxide concentration (e.g. about 400 ppm or about 0.04%) hinders all attempts to sequester the carbon dioxide;

(14) the method sequesters carbon in a form that is not permanent or which will not remain sequestered for more than about a millennium;

(15) the typical existing anaerobic digestion processes anticipate mesophilic or thermophilic anaerobic digestion, and neither temperature range applies to the deep ocean digester; and

(16) the carbon sequestering process discloses requires significant amounts of organisms which can be non-native additions to local ecosystems in many locations, resulting in the need for transport as well as a potential for environmental damage in the event of leakage.

Therefore, there exists a need for cost effective, off-shore energy production technologies with a sustainable capacity to wholly or substantially provide the world's energy needs. Furthermore, such technologies should be self-sustaining and should limit the release of carbon dioxide and other green house gases into the atmosphere.

### SUMMARY OF THE INVENTION

Briefly, and in general terms, the present invention is directed to aquatic energy production systems that produce energy sources via anaerobic digestion. In particular, the present invention relates to off-shore energy production systems containing one or more flexible, inflatable containers supported by water.

Off-shore energy production facilities supported by water bodies may offer many advantages over land-based digesters, including the use of large, available open water bodies as a cost-effective means of support and the potential for locating the facilities at sites that may already contain, or can be easily modified to generate, sufficient amounts of feedstock onsite in a sustainable manner. In addition, the containers and accessory components of the present invention can also be relatively easy to manufacture and ship to remote production sites.

In one of several aspects, the invention relates to an aquatic anaerobic digestion system for producing energy sources. Embodiments of the system can be configured in a variety of ways. The system may contain one or more flexible chambers or containers and plant or animal feedstock material, and the energy sources produced can be renewable, such as methane. At least one container in the system may be an anaerobic digester which holds bacteria that produce energy sources such as methane or hydrogen from aquatic plants. The chambers in the system can be supported by floating in a water body, being suspended in a water body or resting on the floor of a water body. The plant or animal material feedstock may include plant or animal tissue. The system may also include one or more additional chambers and associated mechanisms for purifying methane or sequestering liquid carbon dioxide.

Another aspect is an aquatic method for producing one or more energy sources which may include the anaerobic digestion of plant or animal material within one or more flexible containers and, optionally, substantially purifying the energy source. Embodiments of the method include with out limitation an embodiment which could be configured for off-shore use.

Yet another aspect is a method for sequestering a quantity of carbon dioxide that may include providing and collecting liquid phase carbon dioxide in an energy production system, optionally purifying the carbon dioxide collected, and transporting the some or all of the carbon dioxide to a position on or near the floor of a water body. In some embodiments, the method can be aquatic and the carbon dioxide can be collected in a container for transport.

In a further aspect, a method for sequestering carbon dioxide may include anaerobic bacterial digestion of at least one plant or animal feedstock material in one or more flexible aquatic containers to provide carbon dioxide, substantially purifying the carbon dioxide by separating a liquid phase carbon dioxide from at least one other liquid, and sequestering some or all of the purified carbon dioxide by transporting it to a position on a floor of the water body. The purified liquid may be collected in one or more containers prior to transport in some embodiments, and/or the method can be aquatic.

In yet another aspect of the invention, a method for increasing the density of solid aquatic plant material in a liquid or slurry by transporting the liquid into or through a porous container can be provided. In some embodiments, the aquatic plant material may contain plankton and/or the porous container can be a tube or bag.

These and other aspects and advantages of the invention will become apparent from the following detailed description and the accompanying drawings, which illustrate by way of example the features of the invention.

### BRIEF DESCRIPTION OF THE DRAWINGS

FIG. 1 is a schematic transverse cross sectional view of one embodiment of the invention, a water supported anaerobic digester 10;

FIG. 2 is schematic transverse cross sectional view of one embodiment of the invention, a water supported anaerobic digester 20 configured to produce methane and, liquid carbon dioxide;

FIGS. 3A-3C is a schematic transverse cross sectional view of three embodiments of the invention having different mooring configurations for a water supported digester 30;

FIG. 4 is a schematic transverse cross sectional view of one embodiment of the invention having a water supported digester 40, methane purification chambers 90 and 110, and a nutrient recycling chamber 150;

FIG. 5 is a schematic transverse cross sectional view of one embodiment of the invention having a water supported anaerobic digester 50 with secondary liquid carbon dioxide capture chamber 120 and mixing gas generator 140;

FIG. 6 is a schematic transverse cross sectional view of one embodiment of the invention having a water supported anaerobic digester 60, a methane purification chamber 90, and a liquid carbon dioxide capture chamber 120;

FIGS. 7A-7F is a series of schematic transverse cross sectional views of one embodiment of the invention showing the deployment and recycling of a water supported anaerobic digester 70;

FIG. 8 is a schematic transverse cross sectional view of one embodiment of the invention, a filter tube 81 useful for concentrating a feedstock slurry 82b or liquid 82c; and

FIGS. 9A-9D are a series of schematic transverse cross sectional views of one embodiment of the invention illustrating an approach for employing filter pellets 132a to increase the solids content in a water supported anaerobic digester 130.

### DETAILED DESCRIPTION OF THE INVENTION

The following description presents preferred embodiments of the invention representing the best mode contemplated for practicing the invention. This description is not to be taken in a limiting sense but is made merely for the purpose of describing the general principles of the invention whose scope is defined by the appended claims.

The present invention provides aquatic energy production systems and methods that can produce energy sources via anaerobic digestion, including off-shore energy production systems that produce energy sources such as methane or hydrogen. The systems may contain one or more flexible, inflatable containers supported by water, at least one of which holds bacteria capable of generating an energy source from aquatic plants via anaerobic digestion. In some embodiments, the systems and processes can be uniquely sustainable in that they may simultaneously produce: (1) renewable natural gas from plankton; (2) liquid carbon dioxide for sequestering in the sea floor (thereby reduce greenhouse gases); and (3) recycled nutrients for production of more plankton. In contrast to the typical existing technologies for reducing greenhouse gases, which either require energy to capture and sequester carbon dioxide or produce energy without sequestering carbon dioxide, the systems and methods of the present some embodiments of the present invention can potentially produce up to about 60% of world energy demand while requiring only about 6% of Earth's water surface area to grow plankton. Furthermore, in some embodiments the systems and methods of the present invention can also capture atmospheric carbon dioxide and store it within a tiny fraction of Earth's seafloor sediments where it can remain safely sequestered for many millennia, typically without expending much energy in the process relative to typical sequestration technologies.

Off-shore energy production facilities supported by water bodies can offer many advantages over land-based digesters, including without limitation large, available open water bodies as an alternative means of support, access to a wide range of pressure and suitable temperatures with good heat transfer and specific heat properties, and the potential for locating the facilities at sites that already contain, or can be easily modified to generate, sufficient amounts of feedstock onsite.

Other features and advantages of the invention will be apparent from the following detailed description when taken together with the drawings, and from the claims. The following description presents preferred embodiments of the invention representing the best mode contemplated for practicing the invention. This description is not to be taken in a limiting sense but is made merely for the purpose of describing the general principles of the invention whose scope is defined by the appended claims.

Before addressing details of embodiments described below, some terms are defined or clarified. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The following definitions refer to the particular embodiments described herein and are not to be taken as limiting; the invention includes equivalents for other undescribed embodiments.

As used herein, the term “aquatic” is intended to mean taking place in or on water, including without limitation operating, living, frequenting, or growing in or on water.

As used herein, the term “aquatic plant” is intended to mean any type of plant adapted to living in or on aquatic environments, including without limitation algae, phytoplankton, water hyacinth, duck week, sea kelp and sargassum. Aquatic plants typically grow in standing water and can, for example be found submerged, with floating leaves, or emergent such as rooted beneath the water surface, but growing above it.

As used herein, the term “automated” when referring to a system is intended to mean that the system, or one or more components thereof, may be automatically controlled by means that take the place of human labor, including without limitation mechanical, electronic, or communication devices, such as feed-back loop, feed-forward loop, and automation expert systems for measuring the properties of systems and for the purpose of operation, preventive maintenance, and research.

As used herein, the phrase “body of water” or “water body” is intended to mean any significant accumulation of water covering the Earth or another planet, including without limitation an ocean, a lake, or a river. A water body or body of water can be a naturally occurring geographical feature or a man-made or artificial feature, such as a pond, lake or harbor.

As used herein, the term “digest” is intended to mean to soften, decompose, or break down, including without limitation to digest using by heat and moisture, biological, or chemical action.

As used herein, the term “digester” is intended to mean a vessel, container, or chamber for digesting organic material, including without limitation plant or animal materials.

As used herein, the term “flexible” is intended to mean materials or structures which are not rigid or constricted, including without limitation materials or structures which are elastic or otherwise capable of ready change or easy expansion or contraction. A material that is flexible under some conditions can be rigid under other conditions.

As used herein, the term “incidental animal” is intended to mean animals whose procurement for feedstock purposes occurs merely by chance or without intention or calculation during the course of local operations such as off-shore activities, including without limitation coccolithophorids, jelly fish, insects, sharks, and marine mammals.

As used herein, the term “animal material” is intended to mean material inherent, intrinsic, or incidental to an animal including without limitation animal tissue and by-products consumed, synthesized or released by a living or deceased animal.

As used herein, the term “plankton” is intended to mean the passively floating or weakly swimming, and usually minute, animal and plant life of a body of water.

As used herein, the term “plant material” is intended to mean material inherent, intrinsic, or incidental to a plant including without limitation plant tissue and by-products which are consumed, synthesized or released by a living or deceased plant.

As used herein, the term “tissue” is intended to mean an aggregate of cells, usually an aggregate of a particular kind of cell, that form one of the structural materials of a plant or an animal, together with their intercellular substance, and excluding waste matter discharged from the body.

As used herein, the term “feedstock” is intended to mean raw material supplied to a device, machine, process, or processing system, including without limitation an anaerobic digester.

As used herein, the term “off-shore” is intended to mean located or positioned at a distance from the shore, including without limitation seaward or away from a lake shore.

Attention is now directed to more specific details of embodiments that illustrate but not limit the invention.

The present invention can provide technologies and methods for generating significant quantities of renewable energy sources from organic materials via anaerobic digestion and, optionally, for sequestering green house gases produced as a by-product of the digestion process.

In particular, the present invention can utilize a flexible, aquatic digester to generate renewable methane from the anaerobic digestion of plant and animal material. One embodiment of the present invention can simultaneously either remove unwanted nutrients from water bodies or recycle those nutrients for sustainable algae production. In another embodiment, optionally, the invention may deliver greater than about 99% (99+%) pure methane, without the approximately 40% carbon dioxide that typically accompanies anaerobic digestion, and without parasitic energy loss. In yet another embodiment, as a further option, the invention may convert the produced carbon dioxide into a liquid which can then be available for sustainable sequestering in many ways, including without limitation in containers that lie on, in, or under seafloor sediments.

Anaerobic digestion is a biological process that occurs when organic matter is deprived of oxygen. It is a very robust process occurring over a wide temperature range. For the purposes of the present invention, anaerobic digestion may occur over the full range of ocean temperatures, typically from the about 3° C. of the deep ocean bottom to the more than about 35° C. of the tropical ocean surface.

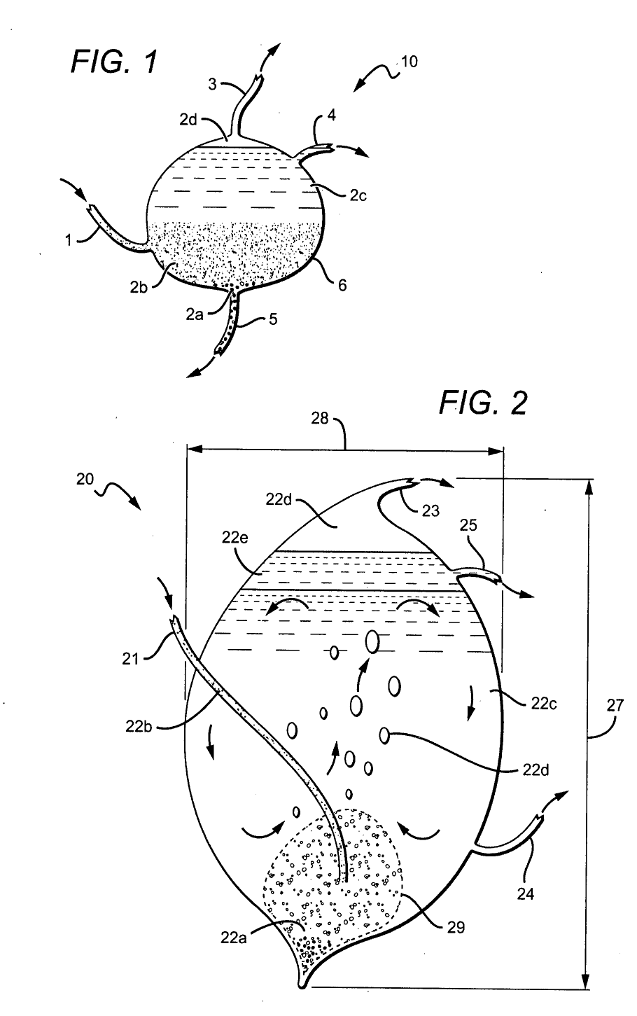

FIG. 1 illustrates the typical features possessed by a system or facility employing anaerobic digesters. The digester 10 is usually an enclosed or substantially enclosed container having sheathing or casing (skin) 6 forming the outside surface of the structure. The digester 10 typically contains one or more materials such as solids 2a, heterogeneous solutions such as slurries 2b, homogeneous solutions or liquids 2c, or gases 2d, including without limitation the following materials:

(a) ambient water, typically local water having dissolved or suspended constituents;

(b) feedstock, typically organic matter such as human waste, algae, plankton or the like. The feedstock may be composed of solids (as at a landfill) or thin slurry. Current terrestrial wastewater treatment facilities, for example, generally send a slurry of about 3% to about 5% solids to the digester 10;

(c) bacteria of a type adapted for gas production using the expected feedstock and conditions. Although naturally occurring, the bacteria may be cultured to increase their numbers within the digester 10;

(d) solid by-products of the bacterial decomposition.

(e) plant growth nutrients, including soluble by-products of bacterial decomposition;

(f) soluble (solubilized) methane or carbon dioxide gas;

(g) gaseous methane or carbon dioxide; and

(h) liquid carbon dioxide.

The proportions of these materials vary during the digestions process. The digester 10 typically also has several input/output means for adding or removing materials from the digester 10 before, during, or after processing. These include without limitation input ports 1 to feed organic matter (e.g. feedstock), usually in the form of a slurry 2b, to the digester 10; output means 3 to recover/capture gas 2d or allow it to escape; output ports 4 to remove/recycle excess water or bulk liquid 2c (e.g. landfill leachate or wastewater facility decant); and output means 5 to address undigested solids 2a (e.g. which may be left in the container/landfill or pumped out to recycle/dispose, as for biosolids from an typical existing wastewater treatment facility). A digester 10 may also contain one or more additional output ports, including recirculation ports (not shown in FIG. 1) to transport bacteria or feedstock which may be removed from the process container during the release of fluids or gases) back to the container for further processing.

When constructing terrestrial anaerobic digesters, there is interest in achieving a higher rate of digestion due to the value of ground space and the expense of the container. The walls of an aboveground container are stressed in a manner proportional to the height of the contained liquid, with higher pressures and stresses at the bottom of the liquid. The stress distribution is a consequence of the difference in density between the liquid inside and outside the container, and the stress must be countered with the added expense of using stress resistant materials in the walls of above ground digesters. The walls of a below ground containment are similarly stressed, although the earth provides a balancing force where the stresses can be countered by expensive excavation or stress resisting materials. Collapsible tanks, which have also been employed as pond liners/lids and suggested for aboveground digesters, present similar stress, space, and expense issues.

The types of support needed to alleviate such stressors for digesters placed above or in the ground are therefore costly and require significant amounts of space. However, by suspending a container of fluid in an ambient fluid of similar density, the skin stress (i.e. stress on the sheathing or casing forming the outside surface of a structure) can be nearly eliminated. If a minor stress is desired for maintaining a desired shape, the stress would be nearly uniform along the height (or depth) of the container. Note that with exactly equal density inside and out, the container would have no skin stress. Slight density differences cause the container to either float or sink. When the container reaches either the surrounding liquid's surface or floor, or it is prevented from sinking or rising, it would tend to spread out, introducing stresses in the skin that would be proportional to the inside-outside density difference. If the container achieves neutral buoyancy, balancing a modicum of gas against a higher than ambient density material in the bottom of the container, the container skin would likely experience vertical stress because the top of the container would be pulling against the bottom of the container. The skins of aquatic digesters could therefore be significantly thinner than those required on land.

The present invention proposes an anaerobic digestion container that is relatively inexpensive for the volume. The flexible digester is suspended in a body of water in order to maximize methane production while minimizing both the expense of the container (by minimizing skin stress) and the use of significant areas of land. A water-supported digester can be relatively large, and may be installed at costs competitive with underground aquifer digesters, but in more locations and with easier access. The invention therefore achieves economic energy source production with an inexpensive but extremely large volume.

A digester of the present invention could contain a mix of organic material and water that is close to the same density as the ambient water. If the ambient water is fresh water with a density close to 1,000 kg/m³, the inside could contain fresh water with the organic material. The density of the resulting slurry would differ only slightly from that of the ambient water. If the ambient water is seawater with a density close to 1,030 kg/m³, the inside could contain seawater with the organic material. Again, the density of the resulting slurry would differ only slightly from that of ambient seawater. In some cases, the inside water may be a blend of fresh water and organics that is even closer to the same density as seawater.

In one embodiment, a thin skinned digester (TSD) digester with volume greater than about 1,000,000 m³ could be made with the same thin plastic currently employed for 0.1 m³ trash bags (i.e. about 0.02 mm thick). The skin may also be thicker, perhaps as much as that employed for landfill lining, including without limitation a skin about 2 mm thick. Alternatively, the skin may be a composite of coated fabric, as is currently employed for collapsible water and oil tanks. The fabric may be any strong fiber, including without limitation nylon, Dacron, fiberglass, HDPE carbon, aramid, cotton, jute, or the like. Typical coatings include vinyl, PVC, HDPE or the like. The skin may also be differently constructed layers of the same material; a thin sheet of HDPE inside a woven HDPE such as that found in the Miratech Geotube® (Commerce, Ga.), for example. The scale of exceptionally large digesters can render even materials normally considered “stiff”, such as steel or reinforced concrete, substantially or totally flexible. For example, a kilometer of steel drill pipe dangling beneath a drill ship can behave more like flexible rope than rigid pipe under such conditions. Larger inside/outside density differences may also be accommodated, but only with proportionally greater stresses, requiring a stronger skin that may be thicker or a stronger material.

Also, leaks are much more of a concern when dealing with waste than with energy production according to the present invention. In one embodiment, the digester could be filled with organic matter that grows, dies, and decomposes in the body of water naturally, rather than with waste. Algae, for example, typically bloom and then decompose in their typical life cycle. When algae decompose in the natural environment, bacteria absorb any dissolved oxygen, sometimes creating “dead zones,” and perhaps even producing carbon dioxide and methane. A “leak” of the contents of an algae filled digester would therefore be in keeping with natural processes. In other embodiments, two or more skins can prove useful for leak prevention and detection.

Furthermore, returning the nutrients produced by an algae fed anaerobic digester back to the ocean surface is important to the sustainability of both the methane production and the carbon sequestering. Algae require nutrients to grow and, in a natural state, the nutrients for growing algae remain in the water or settle slowly to the bottom of the water body. Problems can arise when this natural state is perturbed, such as when humans remove all the nutrients from the water or add unnatural nutrients (e.g. fossil fuel derived fertilizers and fecal matter from unnaturally high density animal/human populations), resulting in decreased fish population and reduced biodiversity. Therefore, unsustainable algae growth and decay (e.g. as a means to sequester carbon dioxide), or unsustainable algae growth and harvesting, should ideally be avoided.

FIG. 2 illustrates some of the special features possessed by one embodiment of the invention, a system or facility employing a large anaerobic digester in the ocean. The digester 20 has a vertical extent 27 on the order of greater than about 500 meters, a horizontal extent 28 (typically a diameter) on the order of greater than about 100 meters, and a volume on the order of 20 million cubic meters. The top of the digester is at a depth of about 500 meters in this embodiment. A filter bag 29 captures feedstock and bacteria arriving via port 21 as slurry 22b and retained in the filter bag as a material with higher solids content 22a, while some excess water 22c exits through output port 24. Bacterial decomposition in the absence of oxygen (anaerobic digestion) will saturate the liquid throughout the digester with carbon dioxide and methane. Unsaturated methane gas 22d can bubble toward the top of digester 20, establishing an upwelling current over the areas of most rapid gas production and, at any depth and temperature, the displaced bulk liquid 22c will produce equal down draft currents in the digester 20, as indicated by the arrows pointing respectively upwards (towards the top of the digester) and downwards (towards the bottom of the digester) of the liquid 22c in FIG. 2. These currents are useful for mixing digester contents such as bacteria and nutrients. Below certain depths and temperatures, the reducing carbon dioxide solubility with decreasing pressure in the upwelling current can also cause the soluble carbon dioxide to come out of solution as a liquid 22e with density on the order of about 90% relative to that of seawater, such that it can collect as a separate layer above the rest of the liquid 22c in the digester 20. At carbon dioxide saturation of the digester liquid 22c, additional bacterial carbon dioxide production will be as a liquid phase that will rise up above the digester liquid 22c. The liquid carbon dioxide 22e may be extracted via port 25 for sequestering. By comparison, at sea level, gaseous carbon dioxide density is only 0.2% of seawater. With increasing pressure, gaseous carbon dioxide compresses to on the order of about 8% that of seawater, just before it becomes a liquid.

There is a multitude of depths, sizes, and temperatures for a digester 20 according to the present invention and not all can produce liquid carbon dioxide. Carbon dioxide is a liquid at pressures as shallow as about 400 meters at about 5° C. or temperatures as high as about 30° C. at about 630 meters. The density of carbon dioxide increases with pressure, becoming denser than seawater at about 3,000 meters such that it can pool on an ocean floor. The density of seawater increases slightly with depth from about 1025 kg/m³ at the surface to 1,070 kg/m³ at 10,000 meters. Liquid carbon dioxide would, for example, be denser than seawater at 5° C., 1,040 kg/m³ at 3,000 meters and 1,130 kg/m³ at 10,000 meters. Table 1 shows several exemplary temperature and pressure conditions under which carbon dioxide changes phase from gas to liquid.

TABLE 1
Temperatures and Pressures at which
CO₂ changes from gas to liquid

| | | | Interpolated | |
|---|---|---|---|---|
| | Pressure to | Depth to | liquid | Calculated gas |
| Temperature | be liquid | be liquid | density | density |
| (deg C.) | (atm) | (meters) | (kg/m³) | (kg/m³) |
| 0 | 34 | 345 | 920 | 70 |
| 5 | 39 | 395 | 880 | 80 |
| 10 | 44 | 444 | 850 | 90 |
| 15 | 49 | 492 | 820 | 100 |
| 20 | 54 | 539 | 790 | 110 |

There are also many ways to operate an anaerobic digester. In one embodiment, additional carbon dioxide could be captured as a liquid when a batch process is employed with internal circulation, as shown in FIG. 2. The solubility of carbon dioxide could be on the order of about 0.055 kg/kg at the bottom of the digester and 0.053 kg/kg at the top of the digester, depending on both depth and temperature. That implies that the digester liquid 22c located near the top of the container, which contains soluble carbon dioxide, could release about 2 kg of liquid carbon dioxide 22e for each 1,000 kg of digester liquid 22c cycled. The batch process would keep the bulk of the digester liquid 22c cycling until the gas 22d production stopped and then the accumulated nutrients in the digester liquid 22c could be recycled for plant growth. In another embodiment, a continuous feed process via port 21 and a continuous output stream via port 24 could be established. The digester liquid 22c expelled via port 24 in this embodiment, could also contain 0.053 kg/kg of dissolved carbon dioxide, which could be captured as a gas along with the 0.001 kg/kg of methane dissolved in the digester liquid 22c. In yet another embodiment, any nutrients and undigested materials in the liquid 22c that are also expelled via port 24 could be recycled back to the digester 10 (not shown in FIG. 2). In another embodiment, one could arrange several TSDs, each operating in batch mode, to provide a continuous supply of methane.

In another embodiment of the present invention, vertical tubes could be provided to contain the upwelling or down draft currents in the digester liquid 22c, as indicated by the respective upward and downward pointing arrows in FIG. 2. The tubes could increase the efficiency (speed) of the currents and provide an opportunity for heat exchange. Because the carbon dioxide solubility also varies with temperature, heating the upwelling current and cooling the down draft would extract more carbon dioxide each cycle. However, warming the carbon dioxide can increase the depth needed for the carbon dioxide to remain a liquid.

There are at several sequestering options for the liquid carbon dioxide, or a portion thereof. In one embodiment, the liquid carbon dioxide could be pumped to depths in excess of about 3,000 meters, where its density will exceed seawater's density. In one embodiment, the liquid carbon dioxide might be injected into the seafloor sediments (aka ooze). The sediment should prevent the liquid from dissolving in the seawater above the sediment. Stable and naturally occurring liquid carbon dioxide has been found in seafloor sediments at a depth of only about 1,500 meters. Note that a pipeline reaching to about 7,000 meters could therefore be density powered because the denser lower liquid could pull down the less dense upper liquid. In another embodiment, such as for additional protection against the possibility of carbon dioxide dissolving back into the seawater, the liquid carbon dioxide could be placed in sealed containers with weights and dropped to the seafloor. The containers could embed deep in the sea floor ooze if provided minor structural integrity and a hydrodynamic shape. Also, a container could glide a long distance to deeper ocean, if provided with hydrodynamic “wings.” In yet another embodiment, the liquid carbon dioxide could be frozen to produce weight and structure. Carbon dioxide's solid density is 1,600 kg/m³. In a further embodiment, the liquid could be reacted to form a solid, such as a stable hydrate that would be denser than seawater and thus could be stored at or near the ocean floor. Many of these embodiments offer the potential for permanent or substantially permanent carbon sequestration, including without limitation sequestration on the order of millennia.

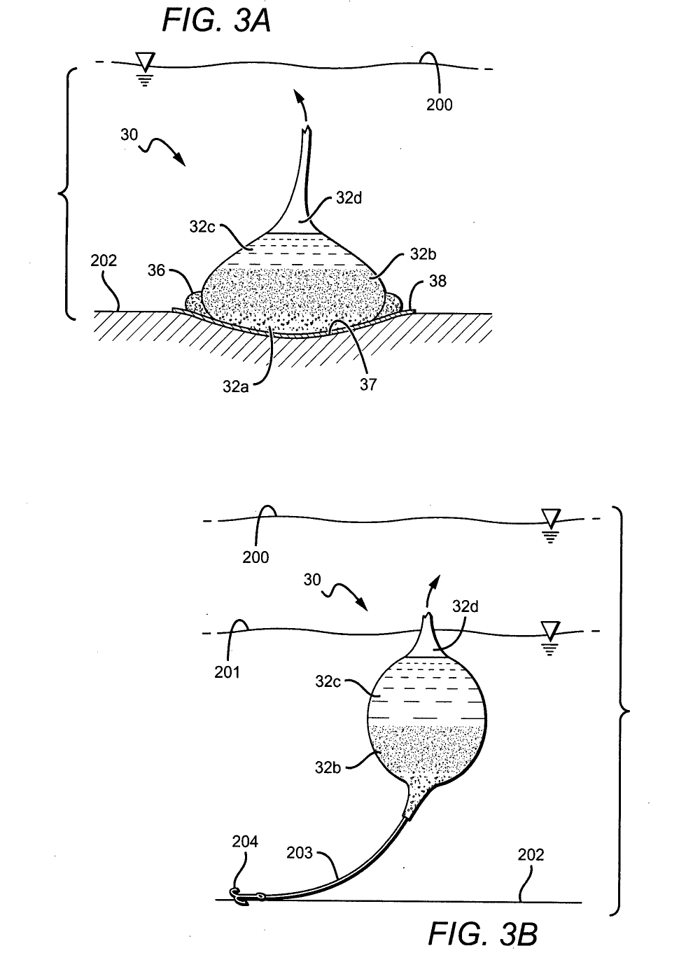

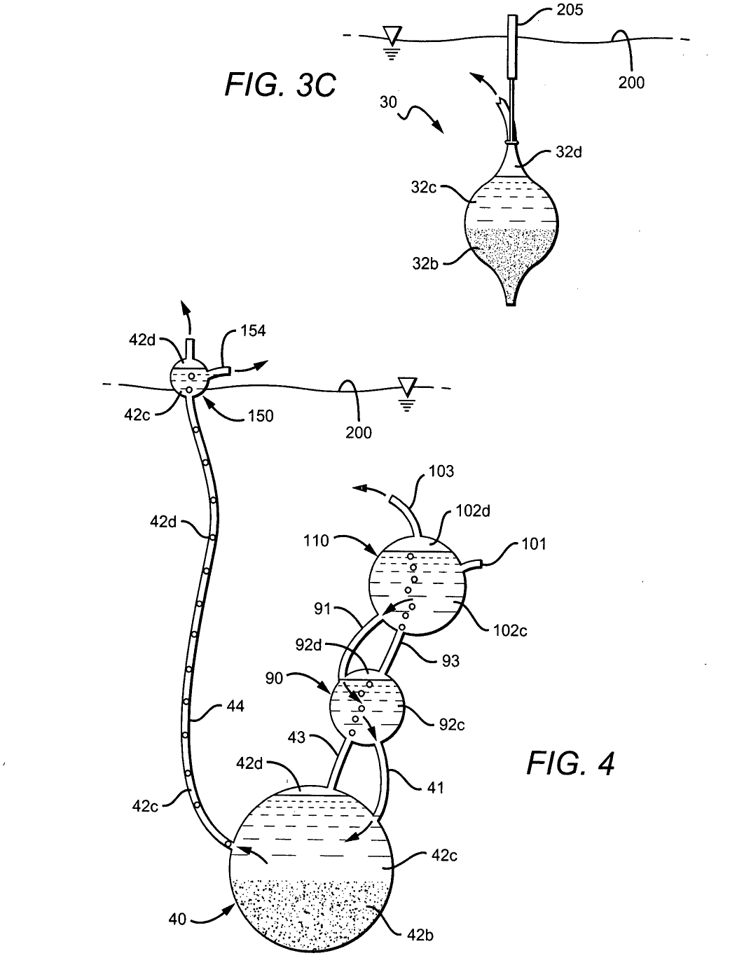

Preferably, a near-surface thin skin digester (TSD) should be substantially or completely flexible in structure, held loosely in position, and allowed to flex and translate with cyclical water motions (e.g. like a jellyfish). With a flexible mooring system, only steady state currents would induce stress in the skin. FIGS. 3A-3C illustrate three basic ways to hold a TSD 30 at a location. In one embodiment, the TSD 30 may rest on the bottom 202 of a pond, lake, sea, or ocean, restrained only by a thin layer of ballast such as a denser-than-water feedstock slurry 32b, or inorganic sediment and solid by-products 32a, as shown in FIG. 3A. The gas 32d provides buoyancy to maintain the structure shape or hold it at the desired depth and the digester liquid 32c can clarify as the slurry 32b settles. An additional ballast pouch 36 containing sediment, or a slight depression 37 underneath the TSD 30, can also be used to prevent rolling. Bottom protection such as a puncture barrier 38 may also be placed underneath the TSD 30. In another embodiment, the TSD 30 might be positioned in the middle of the water column with a mooring that gently resists horizontal currents and any excess of buoyancy, as shown in FIG. 3B. The water level 200 for a submerged mooring would be higher than the water level 201 for a TSD 30 floating on or near the surface in such embodiments. The TSD 30 could also be restrained by a weighted cable 203 or anchor 204. In yet another embodiment, such as when water depths make moorings less economic and distances make moorings less essential (e.g. at a mid-ocean gyre), the TSD 30 might hover, storing and releasing generated gas in order to hover at a desired depth as shown in FIG. 3C. A buoyancy column buoy 205 may be used to assist with depth control.

Surface wave forces in a water body are cyclical and decrease rapidly with depth. Surface wave forces can be avoided entirely by placing the top of the TSD more than about a wave-length deep. That is on the order of about 100 meters in the ocean, much shallower in smaller water bodies. Once below the depth of surface waves, there are few forces that could act on a TSD. Tidal currents are only a major concern where sea bottom contours cause higher velocities. Tsunamis are a concern only very close to the earthquake epicenter and where sea bottom contours focus the wave forces in shallower water. Some ocean currents in some locations have velocities that would induce substantial forces on the TSD, however, if the TSD was prevented from moving with the current.

Turbidity currents, such as an underwater “landslide,” could be a concern for a TSD supported on a sediment-laden slope, which are common in areas where TSDs may be desired. In one embodiment of the present invention, a TSD could float above the sea floor to avoid the turbidity current, as shown in FIGS. 3B-3C. In addition, the moorings in this type of embodiment could be designed to survive or “break-away” during a turbidity current. The break-away would typically require means to reattach before the TSD drifts past pre-positioned replacement moorings. In another embodiment, a TSD designed to “surf” or “water ski” over turbidity currents could rest on a bottom protector 38 designed to plane over turbidity currents. In yet another embodiment, the TSD could simply be disposable.

Other embodiments of the present invention include optional components and methods for separating the methane from the carbon dioxide. The objective is to have a higher fraction of methane in the generated gas (i.e. purifying the gas). In one embodiment, this can be achieved by positioning the digester in relatively deep water. Carbon dioxide is on the order of 10 times more soluble in water than methane (as measured in gas volume or moles), and therefore one can remove liquid carbon dioxide from the methane produced in a submerged digester at the increased pressures inherently present in relatively deep water. A typical digesting slurry (e.g. water and feedstock), for example, preferentially absorbs carbon dioxide at about 2 to about 5 atmospheres, where 5 atm corresponds to the pressure at a depth of about 50 meters. Carbon dioxide solubility increases steeply up to depths of about 500 meters, then less steeply with additional depth. In contrast, methane becomes only slightly more soluble as depth increases. Therefore, at greater depths, a digesting slurry is inherently subjected to a relatively high pressure, and this can be achieved without expending additional energy once the digester is submerged. Furthermore, in other embodiments, one could similarly use the differential solubility between carbon dioxide and methane at greater depths to implement several steps of aqueous carbon dioxide removal, resulting in enhanced methane purification. Increased depth also provides potential energy for gas transmission; most natural gas transmission in the United States is at 500 psi, the pressure of about 340 meters of water (34 atm).

FIG. 4 shows one embodiment of the present invention for producing higher quality (higher purity) methane in a submerged digester 40. This embodiment employs the differential solubility of methane and carbon dioxide at depth, and can be employed at any depth sufficient to arrange a vertical sequence of flexible containers. In this embodiment, bacteria can generate about 600 parts methane and about 400 parts carbon dioxide (mole/mole) in the digester 40. Any liquid materials in the digester 40, including homogeneous liquids 42c such as water and heterogeneous liquids such as slurries 42b, can absorb approximately 10 times (by volume or mole, and varying somewhat with typical ocean depths and temperatures) more of the carbon dioxide present in the liquid relative to methane. As a result, the gas 42d accumulating at the top of digester 40 can be about 570 parts methane and about 100 parts carbon dioxide at depth (about 87% methane). That gas 42d can then be bubbled, via port 43, through a slightly higher water container 90 which can remove 90 parts of carbon dioxide and 9 parts of methane. The gas 92d accumulating in the second stage container 90 can therefore be about 561 parts methane and 10 parts carbon dioxide (already 98% pure methane). An optional third water container 110 could similarly provide gas 102d, via port 103 that is 99.8% pure methane, and additional containers could likewise provide increasing purity. Table 2 illustrates approximate carbon dioxide and methane solubilities at several exemplary depths. Both gases become more soluble with decreasing temperature. Because the availability of data for both gases at the same pressure and temperature is limited, the values shown in Table 2 are interpolations wherever appropriate, based upon existing data. The carbon dioxide solubility data in saline water used for interpolation was taken from published data at temperatures of 10, 20, 30, and up to 100° C. (FIG. 4, B. van der Meer, 2005). The carbon dioxide concentration units in the data were kg/kg. Published solubility data for a 1 mol/kg NaCl solution of methane (Table 5, Z. Duan & S. Mao, 2006) were also used for interpolation. The methane concentration units in the reference were mol/kg for temperatures 0, 30, 60, and up to 200° C. and pressures 1, 50, 100, 150, and up to 2000 bar. There was only one data point for methane at 0° C., and that was at 1 bar, making an interpolation for solubilities at 20° C., 100 meters (10 bar) too speculative for practical use.

TABLE 2
Interpolated solubility of CO₂ and
CH₄ at depth in seawater at 20° C.

| | Depth (meters) | | | | |
|---|---|---|---|---|---|
| Solubility | 0 | 100 | 500 | 1,000 | 2,000 |
| CO₂ (kg/kg) | 0.0015 | 0.014 | 0.049 | 0.055 | 0.06 |
| CH₄ (kg/kg) | 0.00003 | | 0.001 | 0.002 | 0.003 |
| CO₂ (mol/kg) | 0.034 | 0.32 | 1.1 | 1.3 | 1.4 |
| CH₄ (mol/kg) | 0.002 | | 0.07 | 0.1 | 0.2 |

FIG. 4 also illustrates a continuous feed digestion process. The liquid feedstock that enters container 110 through port 101 absorbs carbon dioxide and methane from the gas 92d which is entering container 110 through port 93. The resulting liquid 102c, fully or partially saturated with methane or carbon dioxide, can then move down via port 91 into chamber 90, which is positioned at a greater depth, and then even lower through port 41 into chamber 40, where it can absorb even more carbon dioxide (and probably a little methane) since carbon dioxide solubility increases with depth. The liquid 102c moving down via ports 91 or 41 may require a pump, if suction from the gas-containing liquid rising spontaneously from container 40 into port 44 can collapse the flexible containers 40, 90 or 110.

Nutrient laden liquid 42c in digester 40 may also exit via port 44 and thereafter be conveyed to the surface 200. Any gas 42d, coming out of solution in port 44 may lift the liquid 42c to a level above the ocean surface 200 in a shallow off-gassing chamber 150. Such additional height may be employed to spread nutrients in the liquid 42c via port 154 to areas at or near the water surface 200, or beyond. The gas 42d bubbling to the surface 200 via port 44 could be on the order of 90% carbon dioxide and 10% methane (by volume). This gas may be compressed (not shown) until the carbon dioxide therein liquefies for easier sequestering and the remaining compressed pure methane is captured. Alternatively, the methane and carbon dioxide may be separated using any suitable technology or process, including without limitation membrane or mass difference processes. Optionally, the methane could be flared and the carbon dioxide could return to the atmosphere from whence it came a few months earlier.

Note that seawater containing additional dissolved gas (i.e. fully or partially saturated) is denser than the ambient seawater, but not so dense as to overcome the pumping action of the gas bubbles. Seawater containing more dissolved carbon dioxide is also more acidic, and a stream of more acidic water descending to the seafloor may be locally detrimental. Interestingly, one important result of employing the deep ocean digester can be a net reduction of dissolved carbon dioxide in the ocean leading to a return to pre-industrial pH levels. It is also possible for intense algae farming to temporarily and locally deplete dissolved carbon dioxide, thereby slowing local algae growth.

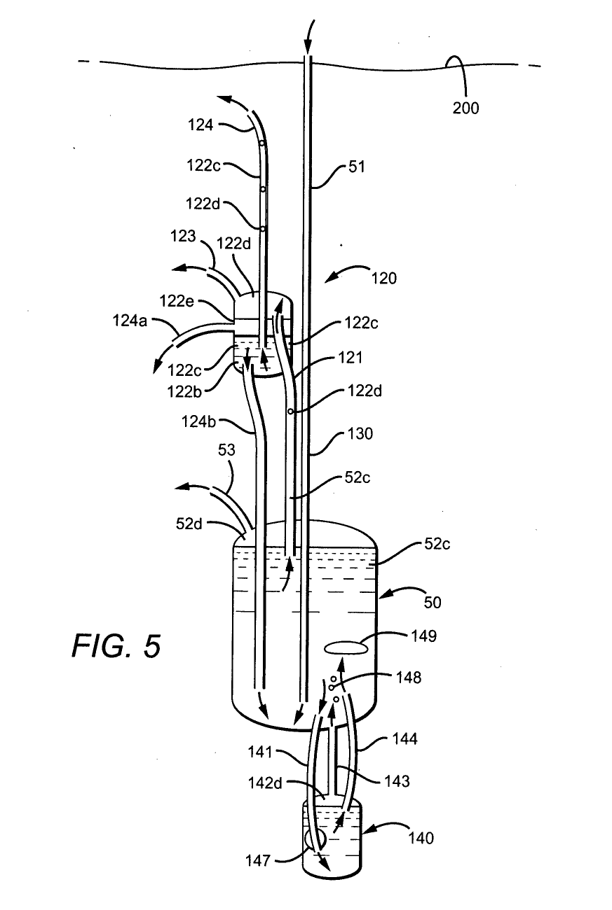

FIG. 5 illustrates another embodiment of the present invention, a system containing a submerged digester 50 for producing higher quality (higher purity) methane (CH₄) and sequestering liquid phase (liquid) carbon dioxide (CO₂). This embodiment benefits from the relative differences in gas solubility for methane and carbon dioxide with both pressure and temperature, as described above for FIG. 4, as well as differences between methane and carbon dioxide with respect to their respective phase change temperatures and pressures for the transition from gas to liquid phase. FIG. 5 illustrates a semi-continuous digestion process using two separate chambers: (1) a main digestion chamber 50 for generating and collecting gases 52d which exit via port 53; and (2) a CO₂ removing chamber 120 in which soluble carbon dioxide comes out of solution as a liquid 122e and collects as a separate layer above the rest of the liquid, as illustrated previously in FIG. 2. The surface 200 water may be from about 10° C. to about 35° C. The CO₂ removing chamber 120 can be on the order of about 500 meters deep where ambient temperatures are generally from about 7° C. to about 14° C. The digester 50 can be on the order of 1,000 or more meters deep. Descending water and feedstock in port 51 can, through a heat exchanger mechanism 130, provide sufficient ambient heat to warm the cool carbon dioxide laden liquid 52c rising from the digester 50 and into the CO₂ removing chamber 120 through port 121. On the way up to and within the CO₂ removing chamber 120, methane gas can come out of solution as a gas 122d and carbon dioxide can come out of solution as a liquid 122e which is less dense than the remaining liquid 122c. Bubbling effects in port 121 resulting from the release of the methane gas 122d and liquid carbon dioxide 122e can “pump” the digester liquid 52c up to the CO₂ removing chamber 120. An optional pumping mechanism could also be used for this purpose if necessary. The gaseous methane 122d and liquid carbon dioxide 122e separated in chamber 120 can be removed via outlet ports 123 and 124a, respectively, and the remaining liquid can be transported back to the digester via an outlet/recirculation port 124b. This continuous feed process has the advantage of recovering methane more rapidly. In another embodiment, the carbon dioxide sequestering process illustrated in FIG. 5 can be paired with the gas purifying process of FIG. 4, such that more methane can be recovered from gas 52d or 122d and more carbon dioxide can be converted to liquid (i.e. and then removed) during processing. Preferably, the circulation rate through the CO₂ removal chamber would be higher than the feedstock input rate.

The CO₂ removing chamber 120 in this embodiment can therefore be maintained with about three layers. The bottom layer may contain slurries 122e or liquids 122c, such as water with digesting feedstock, each of which may also contain dissolved carbon dioxide gas at the equilibrium concentration for the chamber 120 at that particular depth and temperature. The dissolved gases contained in the liquid 122c at the bottom of chamber 120 do not readily come out of solution (as described above for liquid 52c in port 121), and can be readily returned to digester 50 via port 124b. The middle layer can contain the liquid carbon dioxide 122e that came out of solution while rising up to or entering into chamber 120, as described above. The density of this liquid phase carbon dioxide 122e can be about 800 kg/m³ at chamber depths of about 500 meters. The top layer can be nearly pure methane gas 122d, the exact purity depending upon the carbon dioxide phase change equilibrium conditions at the depth of the CO₂ removing chamber 120.

A nutrient-laden fraction of the liquid 122c (e.g. water) in the bottom layer of the CO₂ removing chamber 120 can also be returned to the ocean surface 200 via output port 124, typically at a rate equal to the feed rate of water with feedstock into the digester 50 via input port 51. Such nutrients can be essential for the growth of more algae, which could then in turn be cycled back into the energy production process as feedstock. Bubbling effects in port 124, resulting from the release of gas 122d from liquid 122c within the port, can “pump” the nutrient-laden liquid 122c up to the surface 200. A typical combination of about 90% carbon dioxide gas and about 10% methane gas (by volume) bubbles could pump the water to the surface, for example. Preferable, about 90% of the bottom layer water is recycled back to the digester 50 to help maintain the bacteria population and to improve the amount of methane and carbon dioxide captured per unit of organic matter.

In another embodiment, a second digester 140 acting as mixing gas generator can be placed below the main TSD 50. This mixing gas container 140 could contain pressurized gas that is bubbled into the main digester 50 via port 143, in order to aid in mixing the contents of the main digester 50. The digestion mixture could, for example, typically contain water, feedstock, and bacteria, and an optional pump 147 may be used to assist circulation of the digestion mixture into the mixing gas container 140, via port 141 and exiting again via port 144. The generator 140 in this embodiment is therefore essentially a smaller TSD. Because of the generator's additional depth, the gas 142d that it produces experiences a higher external pressure than the water in the TSD 50. The high-pressure gas 142d can therefore be used mix the bulk contents of the TSD 50, either with coarse bubbles 148 or with large pulsed bubbles 149 such as that produced by a Pulsair™ mixer (Pulsair Systems, Inc., Bellevue, Wash.).

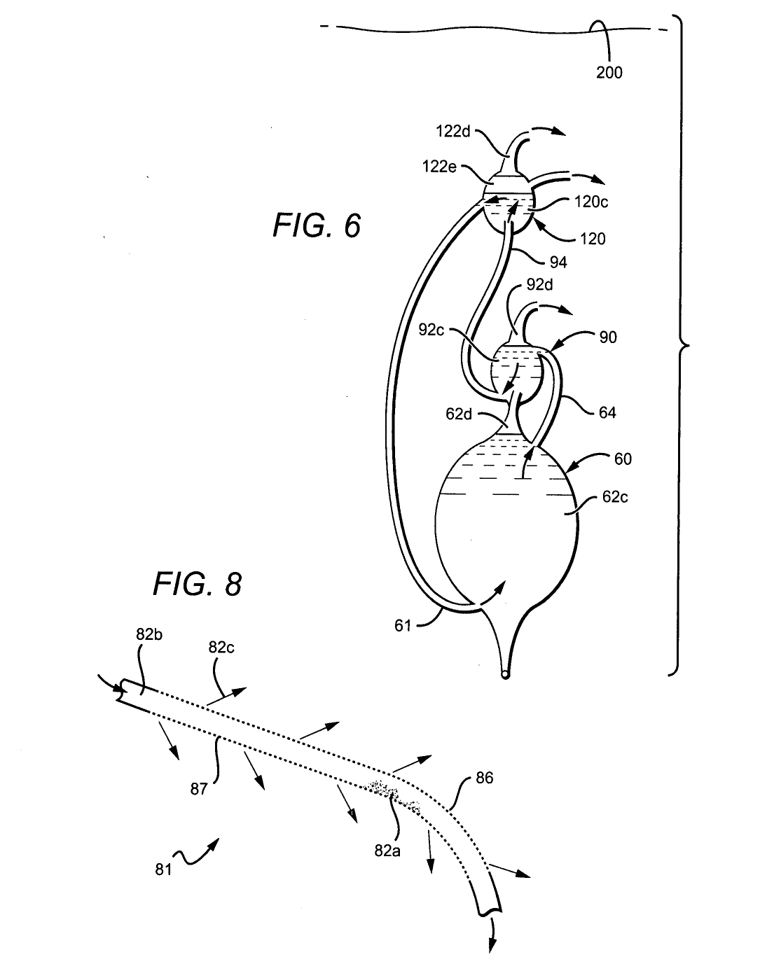

FIG. 6 shows an energy production system having a digester 60 combined with one methane scrubbing (cleaning) chamber 90 and one liquid carbon dioxide separation chamber 120, each of which is at different depths and therefore exposed to different temperatures and pressures. The system is shown with digester 60 and chambers 90 and 120 filled with water, feedstock, and bacteria during a batch process. Digester 60 could operate at 20° C. at a depth of about 1,000 meters. The temperature of the digester 60 may be maintained using a variety. of approaches either in isolation or combination, including without limitation the mass of surrounding water, insulation, and a heat exchanger in contact with warm surface waters. The maximum carbon dioxide solubility could be about 55 parts per thousand (ppt) by weight in the digester 60 and the maximum methane solubility could be about 1.4 ppt under such conditions.

Chamber 90 is also positioned at about 1,000 meters in FIG. 6, but above chamber 10. As the liquid transfers along port 4, it may be chilled from about 20° C. to about 5° C. by employing deeper, colder water and a heat exchanger. In comparison to chamber 10, the maximum carbon dioxide solubility in chamber 90 could improve to about 65 ppt by weight, whereas the maximum methane solubility could be about 1.5 ppt.

Chamber 120 is positioned at about 500 meters deep in FIG. 6. As the liquid transfers along port 94, it may be warmed from about 5° C. to about 20° C. by employing a heat exchanger in contact with more shallow, warmer water. The maximum carbon dioxide solubility in chamber 120 could decrease to about 52 ppt by weight as a result of such warming, while the maximum methane solubility can be about 0.8 ppt under the same conditions. Alternatively, chamber 120 might be positioned slightly shallower or warmer, such that the dissolved carbon dioxide comes out of solution as a gas and the maximum solubility of CO₂ thereby drops to near 40 ppt. Keeping the carbon dioxide gas below about 400 meters and chilling it below about 5° C. would convert the gas into a liquid.

The embodiment illustrated in FIG. 6 is configured as a batch process with an annual addition of feedstock and a continuous, although varying, production of gases 62d, 92d, and 122d as well as liquid CO₂ 122e. Liquid circulates up through ports 64 and 94 and returns to the digester 60 via port 61. In another embodiment, one could arrange several TSDs, each operating in batch mode, to provide a continuous supply of methane.

A water supported TSD and associated containers can be arranged in multiple configurations depending upon the need for energy production, gas separation, and carbon sequestering. The salinity of the water source for the TSD can range from fresh to very salty water, such as that found in the Dead Sea or the Salton Sea. The organic matter used as feedstock can be composed of any suitable material, including without limitation terrestrial brown biofuel plants, terrestrial animal components or excrement, free flowing or fixed aquatic plants, free floating or fixed aquatic animal, or aquatic wastewater. Typically, the methane from a wastewater treatment plant's anaerobic digester will supply on the order of about half the energy required by the wastewater treatment plant. On a global perspective, about 6 billion people produce on the order of about 2 million metric tons of solid waste per day that might be converted, using present waste treatment technologies, to about half that needed to properly treat their waste or about 1% of the world's current energy use. In contrast, the plankton grown on about 10% of the world's ocean surface could sustain production of about 100% of the world's current energy demand using the present invention. Furthermore, the invention's carbon sequestering option allows plankton grown on about 5% of the ocean surface to supply about 50% of the energy needs of the world while allowing fossil fuels to supply the other 50% (because it sequesters the fossil carbon).

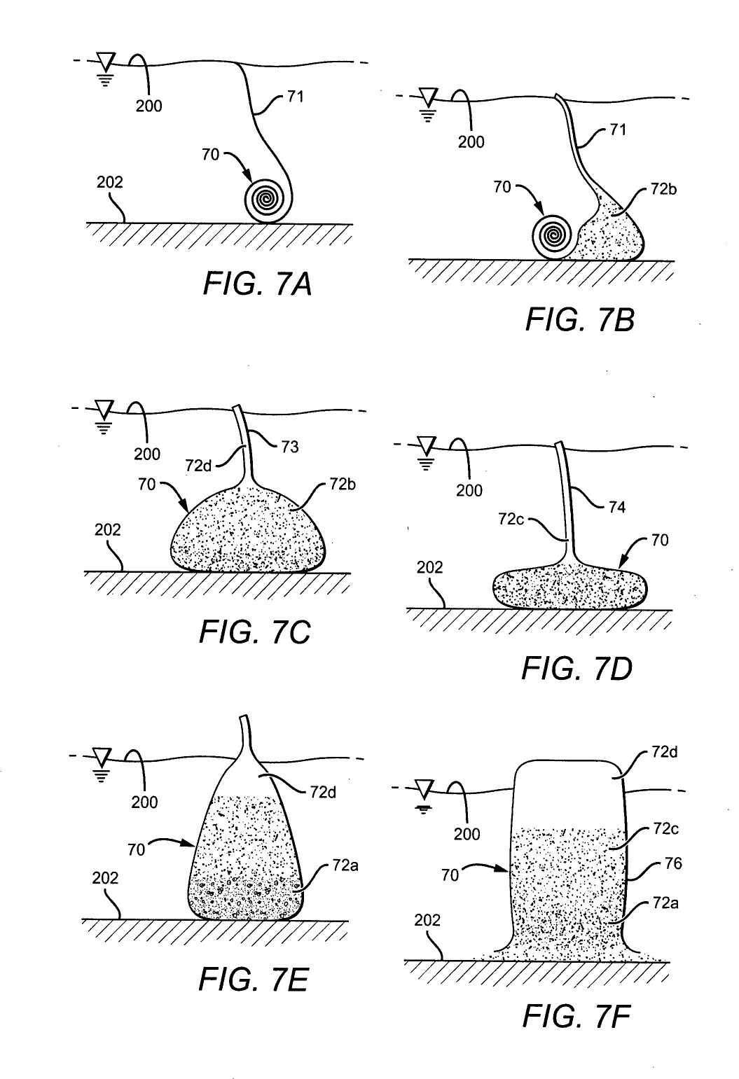

A Thin Skin Digester (TSD) should be relatively easy to manufacture and ship to methane production sites around the world. In one embodiment, the TSD can consist of a roll of plastic carefully packaged, complete with mooring, sensors, pumps, and control systems, such that one can simply drop it off a boat at a remote location and then fill it up on site. The only serious restraint may be the length of pipe that conveys the methane to shore. One simple embodiment of such deployment is shown in FIGS. 7A-7F. A very large, but empty and collapsed TSD 70 is first lowered from the water surface 200 to the water body floor 202, as shown in FIG. 7A. The TSD 70 can then be inflated with an algae-laden liquid (e.g. water) or slurry (feedstock) through a collapsible filling hose 71 as shown in FIG. 7B. As the TSD 70 fills, bacterial decomposition of the feedstock 72b should use up any dissolved oxygen in the TSD 70 and then anaerobically produce methane and carbon dioxide gas 72d. Once the TSD 70 is filled, one can stop adding the algae laden water via the filling hose 71 and continue harvesting methane and carbon dioxide gas 72d from the TSD 70 for some time, as shown in FIG. 7C. The filling hose may then become a gas capture hose 73, as shown in FIG. 7C, or a separate gas hose may be provided. The methods and technologies for operating typical existing anaerobic digesters for by-product gas production, such as limiting the gas pressure so as not to damage the structure, making use of very low-pressure or very high-pressure gas that has a high fraction of carbon dioxide, controlling acid forming and methane producing bacteria, and the like, should translate to the TSD.

When gas production drops below some economic limit, one can either abandon the TSD 70 in-place or recycle materials as shown in FIGS. 7D-F. In one embodiment, the first step in recycling is to remove the nutrient laden liquid 72c in the TSD 70 by temporarily switching port 73 from a gas output port to a liquid output port 74. This can be done after gas production slows down, with the expectation of leaving some solid 72a material behind, including without limitation bacteria-containing feedstock and digested solids. The loss of ballast caused by removing the liquid 72c could then allow any gas 72d subsequently produced to pull the TSD 70 into a vertical position within a few days or weeks, as shown in FIG. 7E. Eventually over time, as shown in FIG. 7F, the oppositional stresses resulting from increasing gas 72d pressure near the top of the TSD 70 and solid accumulation 72a at the base, the bottom of the TSD 70 would tear out of the TSD 70, dropping the remaining bacterial-laden liquid 72c, as well as any solid material 72a, such as digested and stabilized feedstock and solid by-products on the ocean floor, thereby allowing the TSD 70 material to be recovered for later use. One might. For example, recover the TSD skin for reuse and disperse any remaining solids on the water body floor. Alternatively, one may dredge the solids to recycle more nutrients.

The nutrients for growing more algae or for use in terrestrial agriculture are produced by bacteria in the TSD described above. A sustainable operation would include pumping the nutrient laden liquid from the TSD 70 to the water surface 200 and use this effluent to grow more algae locally or move it to land for growing food crops. As a product of the biologic reaction, ammonium will be present at relatively high concentration in the TSD 70 effluent. Metering or otherwise controlling the amount of the nutrient-rich TSD 70 effluent to the surface water or to land should allow sufficient time for the algae to increase the amount of dissolved oxygen in the surface water, thereby allowing the bacteria in the effluent to convert ammonium to nitrate and the algae to absorb the nitrate. A similar process has been employed for wastewater treatment. On land, unsaturated soils contain the necessary oxygen and bacteria for this conversion.

There are many suitable methods for harvesting microalgae, macroalgae, and plankton for use as feedstock, and potentially any means will work with a TSD. Because the TSD is likely to be filled via a collapsible input hose, it may be advantageous to concentrate the organic matter as the matter transits the hose on its way to the digester. The porous tube shown in FIG. 8 is one of many ways to concentrate algae. The porous input tube 81 may be similar to a soaker hose or an open ended Miratech GeoTube® (Commerce, Ga., USA). A dilute slurry solution 82b containing water and feedstock, including without limitation solutions or slurries containing about 0.5% algae and other plankton or pieces of macroalgae, may enter the tube at a rate of about 10 cubic meters per second. As the mixture travels along the tube 81, liquid water 82c can be extruded through the holes 87 in the skin 86 of the porous tube 81. The algae concentration can thereby increase as the mixture moves along the porous tube 81 to enter the digester (not shown), significantly increasing the density of solids in (and therefore the potential energy of) the feedstock. In some embodiments, this approach can increase the feedstock solids density to greater than about 5% algae at 1 cubic meter per second. After use, the tube skin 86 may become plugged with solid material 82a, which may be utilized as additional concentrated feedstock by placing or inverting the tube into the digester (not shown). This approach can therefore provide a method for increasing the solids density and the transportability of plankton slurry by pumping the slurry into a porous container.

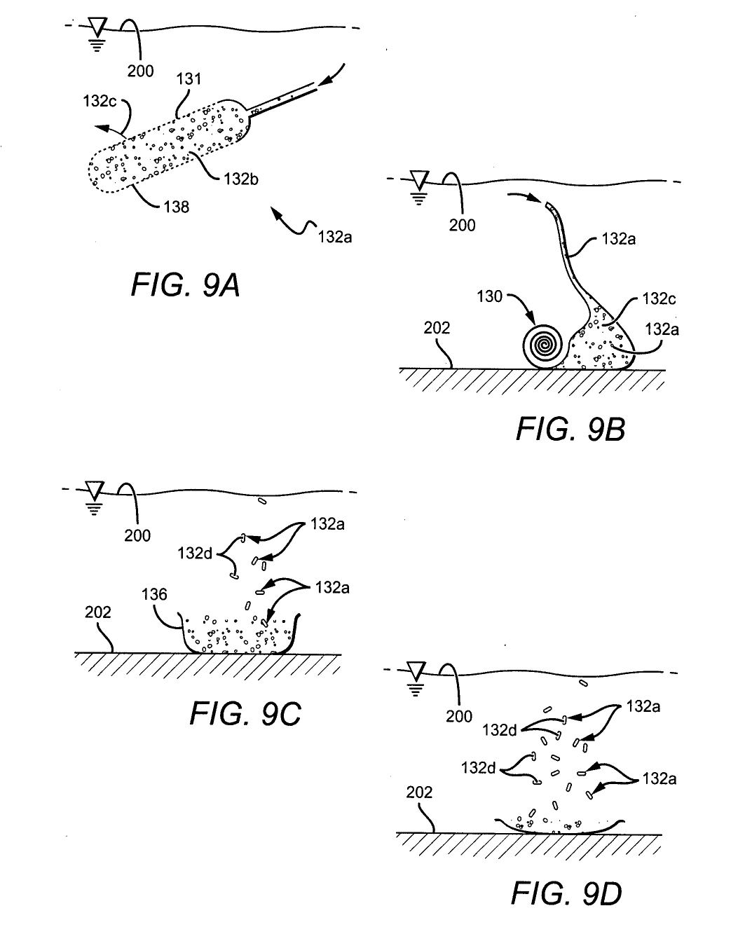

One could similarly form solid pellets 132a using algae pumped into porous filter bags 138 as shown in FIGS. 9A-D. The pellets 132a can be large, on the order of about 1 to about 10 meters in diameter and about 2 to about 50 meters in length. The filter bags 138 may be similar in structure and operation to the Miratech GeoTube® (Commerce, Ga., USA). A dilute slurry 132b of algae feedstock, about 0.5% algae and other plankton or pieces of macroalgae, for example, may first enter the filter bag 138 as shown in FIG. 9A. The excess liquid 132c from the feedstock slurry 132b would exit through the porous skin of the filter bag 138 until the remaining slurry 132c contained more than about 10% by weight of solids. The concentrated solid pellets 132a of organic matter remaining in the filter bag 138 could then be conveyed into a TSD 130 with other filling or input liquid 132c such as water, as shown in FIG. 9B. Inside a filled TSD 130, the higher organic matter densities inside the solid pellets 132a may trap the gases 132d generated inside the pellets 132a as digestion proceeds with time, thereby inducing buoyancy. The bobbing action (not shown) resulting from the buoyant pellets 132a rising, releasing gas, sinking, and rising again, all within the TSD 130, may increase gas production rates.

After the bacteria have converted most of the organic matter in the solid pellets 132a into gas and nutrients, the TSD 130 roof may be opened, as illustrated in FIG. 9C. Measures taken to either capture gas 132d generated within the solid pellets 132a or to inflate a bladder inside the pellets (e.g. for time release fertilizer pellets) could be activated by any suitable means including without limitation devices or mechanisms such as a timer, sensor, or sonar signal. The captured gas 132d or inflated gas bladder could carry the solids arising from the digested pellets 132a, along with nutrients in the pellets, to the water body surface 200, where they could become floating, slow-release, solid fertilizer tablets 132a, as shown in FIG. 9D. After their fertilizer value has depleted, the pellets' porous skin could be inverted for reuse as a new pellet (not shown).

### EXAMPLES

### Example 1

Eutrophied lake revival—Live algae add oxygen to water, while dead algae remove oxygen as they decompose, and eurtrophication is a process by which a body of water becomes enriched in dissolved nutrients (such as nitrates and phosphates) that stimulate the growth of aquatic plant life, usually resulting in the depletion of dissolved oxygen. The algae growing in the lake could be become digester feed and thereby converted to water, methane, carbon dioxide, and fertilizer, and the nutrients captured in the digester could be safely recycled on land agriculture or landscape. Lake bottom nutrients might also be pushed to the surface to speed algae growth and further clean the lake. A moderately eutrophied lake about 1,000-hectares in size could feed a 5,000 m³ digester, which may float or be partially, substantially, or completely submerged. The water supported digester would be collapsible for easy relocation. One might also rotate the digester among several lakes, moving each year, for example. The digester could produce on the order of about 2,000,000 therms of methane a year and annual costs, including loan payments on the digester (after full development), could be on the order of half the value of the methane produced. Returns would probably diminish as the lake becomes cleaner. There are plenty of lakes with approximately 100,000,000 hectares of lake surface in the world. This shallow application produces renewable natural gas with gaseous carbon dioxide, but not liquid carbon dioxide.

### Example 2

Ocean dead zone revival—Similar to the lake situation described in Example 1, but on a larger scale. The Gulf of Mexico dead zone occupies about 2 million hectares, the plankton from which may feed five TSDs, each having a volume of about 1 million cubic meters. The dead zone would then produce about 2 billion therms of natural gas annually. In shallow water (less than about 500 meters), the digester could produce about 90% methane and about 90% gaseous carbon dioxide in two separate gas streams. If the algae dead zone is near deep water, the digester(s) could include the liquid carbon dioxide sequestering option.

### Example 3

Algae farming for renewable methane & carbon sequestering—Similar to the dead zone revival described in Example 2, but over larger areas. An initial dose of nutrients, which may be from deep water, or from the seafloor, could be followed by nutrient recycling to maximize algae production. The harvesting would typically need to be faster than the algae die and decay. There may also be incidental fish production from the algae farm. The Salton Sea Authority, for example, is already harvesting algae (when funding is available) to prevent fish kills resulting from the lack of oxygen when the algae die.

### Example 4

Algae farming for bio-diesel production—In near-shore applications, the TSD could provide a rich source of carbon dioxide to algae bio-diesel growing operations. Algae bio-diesel growth is easier to control with a rich (better than about 20%) carbon dioxide source because ambient air (about 400 ppm carbon dioxide) has other algae and harmful bacteria. The carbon dioxide from the digester gas and methane combustion (e.g. on site electricity generation) could boost oil-bearing algae production and nutrient uptake. After harvesting the algae-oil for bio-diesel, the remaining algae could be digested for methane production.

### Example 5

Dust Control—Lakes and seas are drying up in many places on earth, filling the air with unhealthy dust. For example, the Salton Sea and Dead Sea are both drying; both now lie below sea level, require substantial monetary resources for viability/restoration, and are subject to suggestions of connecting to the ocean. The expense of saving such seas and lakes could be offset by farming algae. The algae can also remove the nutrients that pollute the lake or sea.

### Example 6

OTEC Synergy—An Ocean Thermal Energy Conversion (OTEC) and Deep Ocean Digester may share moorings, vertical water conveying pipes, heat exchangers and an electrical power line. Generating power at the digester could provide energy for freezing carbon dioxide, thereby changing it from liquid to solid phase. Solid carbon dioxide is much denser than seawater, and thus it could be frozen to provide shape and density for sequestering a combination of solid and liquid carbon dioxide deep in the seafloor ooze. Also, an OTEC array combined with an array of thin skin digesters could be used to weaken tropical storms by decreasing ocean surface temperatures. When a tropical storm traverses cooler ocean, it weakens, and when it traverses hotter ocean, it strengthens. Covering select areas of the ocean with OTEC and Deep Ocean Digester may reduce storm surge because both systems tend to cool the ocean surface.

### Example 7

Fresh Water—The TSD may share components with a seawater desalting process, using seawater to produce gas hydrates. In a gas hydrate, water molecules surround a gas molecule, typically six or eight water molecules for each gas molecule. Both methane and carbon dioxide form hydrates at the temperatures and pressures of the deep ocean. When the hydrate is deconstructed, the remaining water has less salt than the original hydrate formation water. The hydrates (also known as clathrates) are solids. Some hydrates are lighter than water, some are denser than water. The “left over” water could have a higher salt concentration and be denser than seawater. The higher salinity water or the hydrate might provide the higher density substance to share containers with, and sink, the liquid carbon dioxide produced by the deep ocean digester. Hydrate formation is exothermic and the heat released may improve the carbon dioxide removal chamber efficiency. In addition, endothermic hydrate deconstruction may aid with converting carbon dioxide from gas to liquid to solid.

The embodiments and examples set forth herein were presented to explain the nature of the present invention and its practical application, and thereby to enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims. For example, while the use of an off-shore anaerobic digester for methane production is described herein, the invention contemplated is not so limited. One skilled in the art will recognize that the invention may potentially be applied in other types of locations which are not off-shore, and could generate energy sources other than methane, for example, without departing from the spirit and scope of the invention as defined in the appended claims.

### REFERENCES

B. van der Meer (2005) “Carbon Dioxide Storage in Natural Gas Reservoirs”, Revue de l'Institut Français du Pétrole, Vol. 60, pp 527-536.

Zhenhao Duan and Shide Mao (2006) “A thermodynamic model for calculating methane solubility, density and gas phase composition of methane-bearing aqueous fluids from 273 to 523 K and from 1 to 2000 bar”, Geochimica et Cosmochimica Acta, Vol. 70, pp 3369-3386.

We claim:

1. A system for producing at least one energy source, the system comprising:
an anaerobic digester, wherein the digester comprises at least one flexible first container supported by a water body; and
at least one feedstock material, wherein the at least one feedstock material comprises at least one of a plant material and an animal material.

2. The system of claim 1, wherein the at least one energy source is renewable.

3. The system of claim 1, wherein the at least one energy source is methane.

4. The system of claim 1, wherein the system is automated.

5. The system of claim 1, wherein the at least one first container is supported by at least one of floating in the water body, suspending in the water body, and resting on a floor of the water body.

6. The system of claim 1, wherein the at least one first container comprises at least one of a gas, a liquid, and a solid.

7. The system of claim 1, wherein the at least one first container comprises at least one bacterium.

8. The system of claim 1, wherein a quantity of nutrients in the at least one first container is transported to the surface.

9. The system of claim 1, wherein the at least one feedstock material comprises at least one plant material comprising plant tissue.

10. The system of claim 9, wherein the at least one feedstock material comprises aquatic plant material.

11. The system of claim 1, wherein the at least one feedstock material comprises at least one animal material comprising animal tissue.

12. The system of claim 7, further comprising at least one flexible second container, wherein:
the at least one second container is coupled to the at least one first container;
the at least one bacterium in the at least one first container produces a quantity of methane in the at least one first container; and
the methane produced in the at least one first container is transported to, and purified in, the at least one second container.

13. The system of claim 12, wherein the methane is purified using at least one differential dissolution property of methane with respect to at least one other gas.

14. The system of claim 12, further comprising at least one flexible third container, wherein:
the at least one third container is coupled to the at least one first container;
the at least one bacterium produce a quantity of carbon dioxide in the at least one first container; and
some or all of the carbon dioxide in the at least one first container is transported to the at least one third container.

15. The system of claim 14, wherein the carbon dioxide transported to the at least one third container is purified by separating a quantity of a liquid phase of the carbon dioxide from at least one other liquid.

16. The system of claim 15, wherein the purified liquid phase carbon dioxide is sequestered by collecting a quantity of liquid phase carbon dioxide and transporting the collected liquid to a position on or near a floor of the water body.

17. The system of claim 16 further comprising at least one fourth container, wherein the purified liquid phase carbon dioxide is sequestered by collecting the quantity of liquid phase carbon dioxide and placing the quantity of collected liquid in the at least one fourth container.

18. An aquatic method for producing at least one energy source, the method comprising anaerobic digestion of at least one feedstock material by at least one bacterium, wherein:
the feedstock material comprises at least one of a plant material and an animal material; and
the feedstock is digested within at least one flexible container supported by a water body, thereby producing at least one energy source; and
optionally, substantially purifying the energy source.

19. An off-shore method for producing at least one energy source according to claim 15.

20. A method for sequestering a quantity of carbon dioxide, the method comprising:
providing a quantity of liquid phase carbon dioxide;
optionally, substantially purifying the liquid phase carbon dioxide;
collecting the liquid phase carbon dioxide; and
placing the collected liquid phase carbon dioxide in at least one storage container, or transporting the collected liquid to a position on or near the floor of a water body.

21. An aquatic method for sequestering carbon dioxide according to claim 20.

22. A method for sequestering carbon dioxide, the method comprising anaerobic digestion of at least one feedstock material by at least one bacterium, wherein:
the feedstock material comprises at least one of a plant material and an animal material;
the feedstock material is digested within at least one flexible container supported by a water body, thereby producing a quantity of carbon dioxide;
substantially purifying the carbon dioxide by separating a liquid phase carbon dioxide from at least one other liquid; and
sequestering the substantially purified carbon dioxide by collecting the liquid phase carbon dioxide in at least one container or transporting the liquid phase carbon dioxide to a position on a floor of the water body.

23. An aquatic method for sequestering carbon dioxide according to claim 22.

24. A method for increasing the density of solid aquatic plant material in a liquid or slurry, the method comprising transporting the liquid into or through a porous container.

25. The method of claim 24, wherein the aquatic plant material comprises plankton and the porous container comprises at least one of a tube and a bag.
